(19) United States
(12) Patent Application Publication
(10) Pub. No.: US 2026/0335310 A1
(43) Pub. Date: Oct. 29, 2026

(54) GEPOTIDACIN FOR USE IN THE TREATMENT OF BACTERIAL PROSTATITIS

(71) Applicant: GLAXOSMITHKLINE INTELLECTUAL PROPERTY DEVELOPMENT LIMITED, Stevenage (GB)

(72) Inventors: Aline BERGESCH BARTH, Collegeville, PA (US); William COUET, Poitiers (FR); Karen O'DWYER, Collegeville, PA (US); Sandrine MARCHAND, Poitiers (FR)

(21) Appl. No.: 19/114,484

(22) PCT Filed: Sep. 25, 2023
(86) PCT No.: PCT/EP2023/076355

§ 371 Date: Mar. 24, 2025

Related U.S. Application Data
(60) Provisional application No. 63/377,064, filed on Sep. 26, 2022.

(51) Int. Cl. A61K 31/4985; A61P 13/08; A61P 31/04

(52) U.S. Cl. CPC A61K 31/4985; A61P 13/08; A61P 31/04

(57) ABSTRACT

The present invention relates to methods for treating bacterial prostatitis, which comprises administration of gepotidacin or pharmaceutically acceptable salts thereof to a human in need thereof.

The project leading to this application has received funding from the Innovative Medicines Initiative 2 Joint Undertaking under Grant Agreement no 853976. This Joint Undertaking receives the support from the European Union's Horizon 2020 research and innovation programme and EFPIA.

### FIELD OF THE INVENTION

The present invention relates to methods of treatment, pharmaceutical compositions, or resistance guided therapies and/or corresponding uses thereof for treating bacterial prostatitis, which comprises administration of gepotidacin or pharmaceutically acceptable salts thereof.

### BACKGROUND TO THE INVENTION

Prostatitis ranges from a straightforward clinical diagnosis in its acute form to a complex, debilitating condition when chronic. It is often a source of frustration for the treating physician and patient. Prostatitis accounts for 8% of visits to urologists, and up to 1% of visits to primary care physicians. In 2000, the estimated cost to diagnose and treat prostatitis was $84 million. Patients with chronic prostatitis often experience impaired mental and physical health-related quality of life. Acute bacterial prostatitis is estimated to comprise approximately 10% of all prostatitis cases.

The challenges of treating bacterial prostatitis with antibiotics are myriad. While the prostate is located near the bladder and urethra, the prostate is not directly connected to the urinary tract through vascular connections. The prostate is also not a highly vascularized organ. Together, this can limit drug exposure to the prostate. Additionally, due to the prostate's anatomical location, it can be difficult to measure drug exposure in the prostate to determine if a drug is adequately reaching the prostate. For at least these reasons, finding effective treatments for bacterial prostatitis can be challenging.

To date, a variety of antibacterial drugs have been developed which have become clinically extremely important antimicrobial drugs. Researchers at GSK described a novel class of antibacterial agents that target type IIA topoisomerases [see Nature, Volume 466, pages 935-940 (19 Aug. 2010) and Gibson et al. Mechanistic and Structural Basis for the Actions of the Antibacterial Gepotidacin against Staphylococcus aureus Gyrase, ACS Infectious Disease, 2019, 5, 570-581] that has shown activity against a broad spectrum of gram-positive and gram-negative bacteria. International Patent Publication WO 2008/128942 and U.S. Pat. No. 8,389,524, hereby incorporated by reference in their entirety, disclose tricyclic nitrogen containing compounds as antibacterial compounds, pharmaceutical compositions and corresponding uses thereof. To date, the utility of gepotidacin in the treatment of bacterial prostatitis has not been shown.

There is a demand for development of effective antibiotics for treating bacterial prostatitis. The present invention is directed to overcoming these and other problems encountered in the art.

### SUMMARY OF THE INVENTION

The present invention provides a method for treating bacterial prostatitis in a human in need thereof, comprising administering to said human a therapeutically effective amount of gepotidacin or a pharmaceutically acceptable salt thereof.

The present invention also provides use of gepotidacin or a pharmaceutically acceptable salt thereof in the manufacture of a medicament for the treatment of bacterial prostatitis.

The present invention also provides a kit comprising gepotidacin or a pharmaceutically acceptable salt thereof for use in the treatment of bacterial prostatitis.

### DESCRIPTION OF DRAWINGS/FIGURES

FIGS. 1A-1D show unbound gepotidacin concentrations in blood, muscle, and prostate tissue in healthy and infected rats. Dots, triangles or squares are means, bars represent ±1 standard deviation. FIG. 1A shows decimal representation of unbound gepotidacin concentrations in healthy rats. FIG. 1B shows semi-log representation of unbound gepotidacin in healthy rats. FIG. 1C shows decimal representation of unbound gepotidacin concentrations in infected rats. FIG. 1D shows semi-log representation of unbound gepotidacin in infected rats.

### DETAILED DESCRIPTION OF THE INVENTION

The terms “antimicrobial”, “antibiotic” and “antibacterial” are used interchangeably in the present application, and refer to any natural or synthetic compound which kills or inhibits the growth of a microorganism.

Further as understood in the present invention, antibiotic resistance occurs when bacteria change in response to the use of antibiotics, making them ineffective; and antimicrobial resistance is a broader term, encompassing resistance to drugs that treat infections caused by other microbes as well, such as parasites (e.g. malaria or helminths), viruses (e.g. HIV) and fungi (e.g. Candida).

Gepotidacin is a first-in-class, novel triazaacenaphthylene antibiotic with the ability to selectively inhibit bacterial DNA replication by a means not utilized by any currently approved human therapeutic agent, therefore providing the opportunity to address an unmet medical need. Gepotidacin and its racemic form is disclosed in WO 2008/128942 (herein incorporated in its entirety). Gepotidacin is (2R)-2-({4-[(3,4-dihydro-2/pyrano[2,3-c]pyridin-6-ylmethyl)amino]-1-piperidinyl}methyl)-1,2-dihydro-3H,8/2a,5,8a-triazaacenaphthylene-3,8-dione:

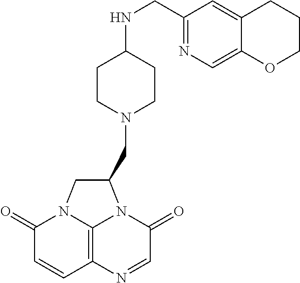 [SMILES: O=c1ccc2ncc(=O)n3c2n1C[C@H]3CN1CCC(NCc2cc3c(cn2)OCCC3)CC1]

The methods and uses of the present invention are based on gepotidacin or a pharmaceutically acceptable salt thereof. Pharmaceutically acceptable salts of gepotidacin include, but are not limited to gepotidacin hydrochloride and gepotidacin mesylate. In an embodiment, the pharmaceutically acceptable salt is gepotidacin mesylate.

International Patent Application Publication No. WO 2021/219637 (herein incorporated in its entirety) describes certain crystalline forms of gepotidacin, including gepotidacin mesylate dihydrate, gepotidacin mesylate anhydrate, gepotidacin mesylate monohydrate and gepotidacin anhydrate (free base). In a first aspect, the present invention provides a method for treating bacterial prostatitis in a human in need thereof, comprising administering to said human a therapeutically effective amount of gepotidacin or a pharmaceutically acceptable salt thereof. In one embodiment, the bacterial prostatitis is acute bacterial prostatitis. In another embodiment, the bacterial prostatitis is chronic bacterial prostatitis.

As used herein, a “human in need thereof” means a person having a prostate, who is diagnosed with prostatitis by a doctor. In one embodiment, the human in need thereof is a male.

Prostatitis is a group of prostate disorders that manifests with a combination of predominantly irritative or obstructive urinary symptoms and perineal pain. (Andriole, G. (2020). Prostatitis. In Merck Manal Professional Version. Merck & Co., Inc. Retrieved Mar. 18, 2021, from https://www.merckmanuals.com/professional/genitourinary-disorders/benign-prostate-disease/prostatitis). Prostatitis can be bacterial or non-bacterial. As used herein, “bacterial prostatitis” refers to prostatitis caused by bacteria. Prostatitis is classified into 4 categories according to the NIH Consensus Classification System for Prostatitis (Krieger, J. N., Nyberg, L., Nickel, J. C. JAMA. 1999; 282 (3): 236-237)—acute bacterial prostatitis, chronic bacterial prostatitis, chronic prostatitis/chronic pelvic pain syndrome; and asymptomatic inflammatory prostatitis. Patients with acute bacterial prostatitis often experience systemic symptoms such as fever, chills, malaise, and myalgias. Chronic bacterial prostatitis manifests with recurrent episodes of infection with or without complete resolution between bouts; symptoms can last for a month or more (e.g., symptoms lasting≥3 months).

As used herein, “prostatitis caused by a bacterium” may mean that the cited bacterium has been identified as being the cause or part of the cause of the prostatitis, or it may mean that the bacterium is suspected or strongly suspected to be the cause or part of the cause of the infection, due to identification of symptoms and other factors such as patient history or local epidemiology. Bacteria which are commonly identified as being the cause of bacterial prostatitis, or strongly suspected to be the cause of bacterial prostatitis, or part of the cause of bacterial prostatitis, include Escherichia coli, Pseudomonas aeruginosa, Klebsiella spp. (including Klebsiella pneumoniae, Klebsiella oxytoca, Klebsiella aerogenes, and Klebsiella variicola), Enterococcus spp. (including Enterococcus faecalis, Enterococcus faecium, and Enterococcus cloacae complex), Proteus spp. (including Proteus mirabilis, Proteus hauseri and Proteus peneri), and Serratia spp. (including Serratia marcescens). Other less common causative bacteria include: Staphylococcus spp. (including Staphylococcus saprophyticus, Staphylococcus lugdenensis, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans and Staphylococcus warneri), Streptococcus spp. (including Streptococcus agalactiae, Streptococcus group F, Streptococcus group G, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitis, Streptococcus oralis, Streptococcus mutans, Streptococcus parasanguinis, Streptococcus salivarius, Streptococcus sanguinis, Streptococcus vestibularis), Mycoplasma genitalium, Neisseria gonorrhoea, Salmonella spp, and Ureaplasma urealyticum. The in vitro efficacy of gepotidacin or pharmaceutically acceptable salt thereof against these and other organisms is shown in, for example, international patent application publication nos. WO2008/128942, WO2016/027249, WO2020/201833 and WO2021/004910. As would be understood by the skilled person, it is envisaged that gepotidacin or a pharmaceutically acceptable salt could be used against bacterial prostatitis caused by any bacterium against which gepotidacin has antibiotic activity.

In an aspect, the bacteria which can be identified as being the cause of bacterial prostatitis, or strongly suspected to be the cause of bacterial prostatitis, or part of the cause of bacterial prostatitis is Escherichia coli.

In one embodiment, for any aspect of the present invention, the human is administered gepotidacin or a pharmaceutically acceptable salt thereof for 1, 2, 3, 4, 5, 6, 7, 8, 9, 10, 11, 12, 13 or 14 days. In one embodiment, for any aspect of the present invention, the gepotidacin or a pharmaceutically acceptable salt thereof is administered at 1500 mg, b.i.d. (total daily dose 3000 mg) for 5 days. In another embodiment, for any aspect of the present invention, the gepotidacin or a pharmaceutically acceptable salt thereof is administered at two doses of 3000 mg each, 6-12 or 10-12 hours apart.

The gepotidacin or a pharmaceutically acceptable salt thereof may be present in a pharmaceutical composition which comprises gepotidacin or a pharmaceutically acceptable salt thereof and at least one pharmaceutically acceptable excipient(s).

In one aspect, the present invention provides a method for treating bacterial prostatitis in a human in need thereof, comprising administering to said human a therapeutically effective amount of gepotidacin or a pharmaceutically acceptable salt thereof, wherein the human has failed at least one prior line of treatment for the bacterial prostatitis. In one embodiment, the method for treating bacterial prostatitis in a human in need thereof comprises administering to said human a therapeutically effective amount of gepotidacin or a pharmaceutically acceptable salt thereof, wherein the human has failed one prior oral antibiotic treatment for the bacterial prostatitis. In one embodiment, the prior oral antibiotic is a fluoroquinolone (e.g. ciprofloxacin, norfloxacin, levofloxacin or ofloxacin), trimethoprim-sulfamethoxazole or doxycycline.

In another aspect, the present invention relates to resistance guided therapy for treating bacterial prostatitis, comprising administering a therapeutically effective amount of gepotidacin or a pharmaceutically acceptable salt thereof.

As would be understood by the skilled person, as used herein, “resistance guided therapy” means a course of therapy, the direction of which is guided by knowledge of the phenotypic or genotypic susceptibility of the microorganism to a given antibiotic, for example as described in Bradshaw et al, The Journal of Infectious Diseases, Volume 216, Issue suppl_2, 15 Jul. 2017, Pages S412-S419. Detecting the causative bacteria in an infection, then detecting resistance of the identified strain to certain antibiotics, in advance or during the course of treatment, has the advantage of potentially reducing the patient's exposure to ineffective antibiotics that may lead to resistance. Identification of a particular pathogen, for example Escherichia coli, may be performed by any suitable genotypic or phenotypic means, such as by NAAT.

Thus in one aspect, the present invention provides a resistance guided therapy for treating bacterial prostatitis caused by Escherichia coli comprising administering a therapeutically effective amount of gepotidacin or a pharmaceutically acceptable salt thereof, to a human in need thereof.

In one aspect, the present invention relates to resistance guided therapy for treating bacterial prostatitis caused by Escherichia coli, where the bacterial prostatitis is acute bacterial prostatitis.

In one aspect, the present invention relates to resistance guided therapy for treating bacterial prostatitis caused by Escherichia coli, where the bacterial prostatitis is chronic bacterial prostatitis.

In another aspect, the present invention provides gepotidacin or a pharmaceutically acceptable salt thereof for use in the treatment of bacterial prostatitis in a human. In an aspect, the bacterial prostatitis is acute bacterial prostatitis. In another aspect, the bacterial prostatitis is chronic bacterial prostatitis. In an embodiment, the bacterial prostatitis is caused by Escherichia coli.

In another aspect, the present invention provides use of gepotidacin or a pharmaceutically acceptable salt thereof, in the manufacture of a medicament for the treatment of bacterial prostatitis in a human. In an aspect, the bacterial prostatitis is acute bacterial prostatitis. In another aspect, the bacterial prostatitis is chronic bacterial prostatitis. In an embodiment, the bacterial prostatitis is caused by Escherichia coli.

In another aspect, the present invention provides a kit comprising gepotidacin or a pharmaceutically acceptable salt thereof, for use in the treatment of bacterial prostatitis in a human. In an aspect, the bacterial prostatitis is acute bacterial prostatitis. In another aspect, the bacterial prostatitis is chronic bacterial prostatitis. In an embodiment, the bacterial prostatitis is caused by Escherichia coli.

In another aspect, the present invention relates to a use of a pharmaceutical composition as defined in the present invention for the manufacture of a medicament for treating bacterial prostatitis, such as acute bacterial prostatitis or chronic bacterial prostatitis.

In another aspect, the present invention relates to a use of a pharmaceutical composition as defined in the present invention for resistance guided therapy for treating bacterial prostatitis caused by Escherichia coli, in a human in need thereof.

### Compounds Used in the Present Invention

WO2008/128942 discloses the preparation of the free base and the hydrochloride salt of gepotidacin.

It will be understood that the phrase “gepotidacin or a pharmaceutically acceptable salt thereof” is intended to encompass gepotidacin, a pharmaceutically acceptable salt of gepotidacin, a solvate of gepotidacin, or any pharmaceutically acceptable combination of these. Thus by way of non-limiting example used here for illustrative purpose, “gepotidacin or a pharmaceutically acceptable salt thereof” may include a pharmaceutically acceptable salt of gepotidacin that is further present as a solvate.

As used herein, gepotidacin or any pharmaceutically acceptable salt thereof may be in any physical form thereof, including non-solid forms such as liquid or semi-solid forms, solid forms such as amorphous or crystalline forms, specific polymorphic forms and solvates including hydrates.

Suitable pharmaceutically acceptable salts include those described by Berge, Bighley and Monkhouse J. Pharm. Sci (1977) 66, pp 1-19.

In one embodiment, in any aspect of the invention, the gepotidacin is gepotidacin free base or is gepotidacin methanesulphonate (mesylate).

The present invention includes within its scope all possible stoichiometric and non-stoichiometric salt forms.

### Pharmaceutical Compositions and Formulations

In particular, gepotidacin or pharmaceutically acceptable salts, used in the present invention may be formulated for administration for use in human or veterinary medicine, by analogy with other antibacterials/antitubercular compounds.

The pharmaceutical compositions used in the present invention may be formulated for administration by any route and include those in a form adapted for oral or parenteral use and may be used in mammals including humans.

The compositions may be in the form of tablets, capsules, powders, granules, lozenges, suppositories, creams or liquid preparations, such as oral or sterile parenteral solutions or suspensions.

In one embodiment, the gepotidacin or pharmaceutically acceptable salt thereof of the present invention is in a tablet or a capsule form. In one embodiment, it is in a tablet form. In one embodiment, the tablet is a 750 mg tablet.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Moreover, the quantity of the compound or pharmaceutical composition used in the present invention administered will vary depending on the patient and the mode of administration and can be any effective amount.

In accordance with any of the methods of administration of the present invention, the term a “therapeutically effective amount”, as used herein, generally includes within its meaning a non-toxic but sufficient amount of the particular drug to which it is referring to provide the desired therapeutic effect.

Treatment regimens for the administration of the compounds and/or pharmaceutical compositions used in the present invention can also be determined readily by those with ordinary skill in art. Where the compositions comprise dosage units, each unit will preferably contain from 50-1000 mg of the active ingredient. Unless otherwise noted, the amount of the active ingredient (i.e., gepotidacin) refers to that of gepotidacin free base.

Depending upon the treatment being effected, the compounds, and/or compositions of the present invention can be administered orally, intravascularly, intraperitoneally, subcutaneously, or intramuscularly. Preferably, the composition is adapted for oral administration. In any of the above aspects of the present invention, in one embodiment, the gepotidacin or a pharmaceutically acceptable salt thereof is administered orally.

It is to be understood that the invention is not limited to the aspects or embodiments illustrated hereinabove and the right is reserved to the illustrated aspects or embodiments and all modifications coming within the scope of the following claims.

The various references to journals, patents, and other publications which are cited herein comprise the state of the art and are incorporated herein by reference as though fully set forth.

The Examples set forth below are illustrative of the present invention and are not intended to limit, in any way, the scope of the present invention.

### Example 1

The example herein describes microdialysis experiments in E. coli infected rats to measure tissue distribution of unbound gepotidacin. In vivo microdialysis experiments simultaneously determined unbound gepotidacin concentrations in rat prostate, muscle, and blood. It is important to understand the tissue distribution of unbound gepotidacin, especially in the target prostate tissue, since gepotidacin must diffuse through capillary membranes to reach the prostate. An assumption that the plasma concentration of gepotidacin represents a suitable surrogate of prostate concentrations, without prostate tissue confirmation, may lead to erroneous conclusions, especially since the pathogen is localized in the prostate.

Unbound gepotidacin concentrations in blood, muscle, and prostate were found to be similar across all tissues, thus indicating that gepotidacin is sufficiently delivered to the prostate and supports the conclusion that gepotidacin is effective in treating bacterial prostatitis.

### Methods

### In Vivo Microdialysis in Rats

An Escherichia coli prostatitis model in rats was obtained as follows. Four days before microdialysis, rats (n=11) were infected with Escherichia coli ATCC 13441 in the prostatic urethra, to induce E. coli prostatitis, at an inoculum of 10⁷ CFU/rat. Healthy rats (n=9) were not infected with E. coli as control.

The day before the experiment, two CMA/20 probes (membrane length 10 mm, Harvard Apparatus, Courtaboeuf France) were inserted into right jugular vein and the right hind leg muscle of healthy and infected rats under anesthesia and analgesic treatment. In parallel, catheters were inserted into the femoral vein and artery to administer gepotidacin intravenously (IV) and to collect blood samples for determination of total plasma gepotidacin concentrations, respectively. The rats were allowed to recover consciousness, and food was withdrawn for 12 hours.

On the day of the experiment, healthy control and infected rats were re-anesthetized and a CMA/20 Elite probe (membrane length 4 mm) was inserted into the prostate. All probes were connected to infusion pumps and perfused with Ringer containing a calibrator (((R)-1-((4-(((2,3-dihydro-[1,4]oxathiino[2,3-c]pyridin-7-yl)methyl)amino)piperidin-1-yl)methyl)-1,2-dihydro-3H,8H-2a,5,8a-triazaacenaphthylene-3,8-dione; 1500 n/mL) at a flow rate of 0.5 μL/min with Ringer. After an equilibration period, gepotidacin was administered by IV bolus at a dose of 20 mg/kg. Blood, muscle, and prostate dialysates were collected every 10 minutes for 90 minutes, and then every 30 minutes for 300 minutes (6 hours). Seven blood samples were also collected for analysis of gepotidacin pharmacokinetics in total plasma. Gepotidacin concentrations were measured in dialysates and in plasma by LC-MS/MS.

### Data Analysis

Pharmacokinetic (PK) parameters were determined in each individual rat by a noncompartmental approach according to standard procedures and with the software Phoenix WinNonLin 7. (Certara, Princeton, NJ) Total unbound body clearance (CLᵤ) was calculated as CLᵤ=dose/AUC_{u,blood}, where AUC_{u,blood} is the total area under the unbound blood concentration-versus-time curve from zero to infinity, equal to the area under the curve from zero to the last measured concentration AUC_{0-last,u,blood} added to the area under the curve from the last measured concentration to infinity AUC_{last-infinity,u,blood}. AUC_{0-last,u,blood} was calculated by linear-logarithmic trapezoidal method. AUC_{last-infinity,u,blood} the area remaining under the curve after the last measured concentration, C(last)_{u,blood} was determined from C(last)_{u,blood}/ke_{blood}. The elimination rate constant (ke_{blood}) and its corresponding half-life (t_{1/2,blood}) were estimated by least squares fit of the data points (log concentration-time). The linear-logarithmic trapezoidal method was used to compute the area under the unbound blood concentration-versus-time moment curve AUMC_{0-last,u,blood} from zero to the last measured concentration. AUMC_{last-infinity,u,blood} the area remaining under the curve after the last measured concentration was determined from: tlast×C(last)_{u,blood}/ke_{blood}+C(last)_{u,blood}/ke_{blood}². The mean residence time from zero to infinity, MRT_{0-infinity} was determined from AUMC_{0-infinity,u,blood}/AUC_{0-infinity,u,blood}. The volume of distribution (VSSᵤ) was obtained from MRT_{0-infinity}×CLᵤ. The AUCᵤ and t_{1/2,u} in tissues were also estimated by the same procedure.

### Results

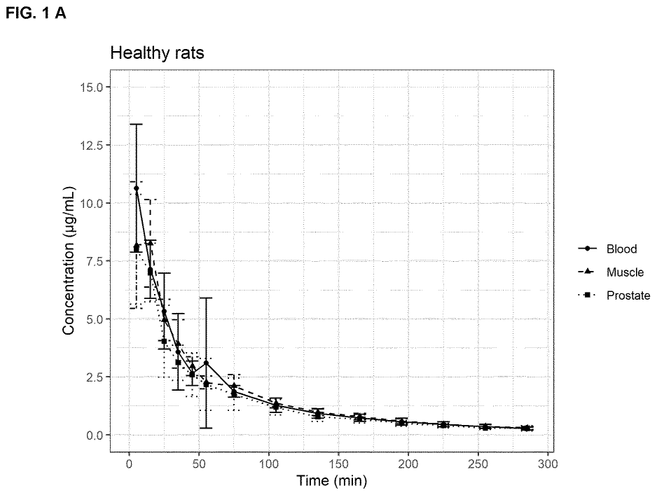

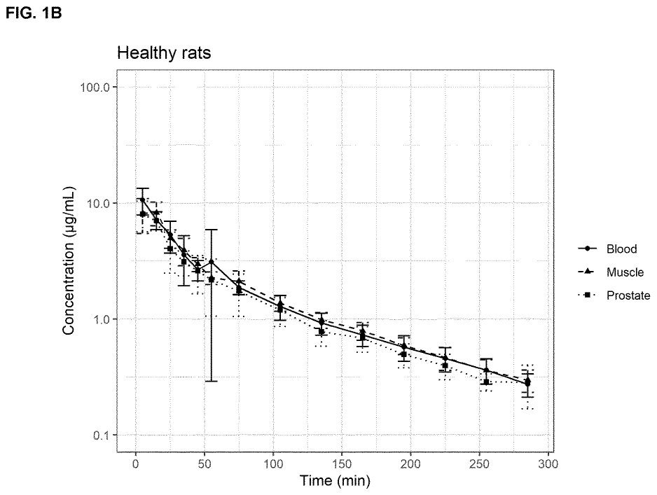

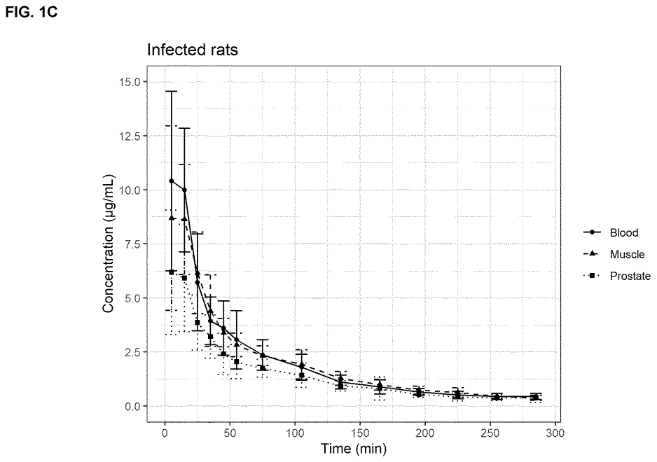

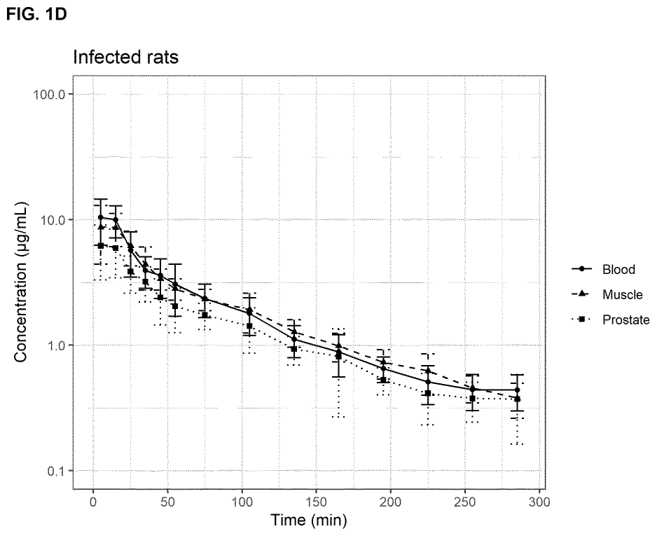

Unbound concentrations of gepotidacin are illustrated in healthy control across different tissue types (blood, muscle, and prostate) in FIG. 1A, 1B. The unbound concentration time profile in blood was biphasic and the concentration profiles in prostate and muscle were almost superimposed with blood concentrations with the exception of the first time point (FIG. 1A). In the prostatitis (i.e., infected) rats, the unbound gepotidacin concentration time profile in blood was also biphasic and gepotidacin concentrations in prostate appear slightly lower than in blood (FIG. 1C-1D). Pharmacokinetic parameter values obtained in the healthy and infected groups are presented in Tables 1 and 2. Gepotidacin half-life and C_{max,u} in blood, muscle, and prostate of healthy control rats were similar (Table 1) and this comparability is supported with AUCᵤ ratios of approximately 1 (Table 2). In infected rats, Cₘₐₓ in prostate were slightly lower compared to healthy rats but gepotidacin half-lives were the same between blood, muscle, and prostate. (Table 1). The muscle/blood and prostate/blood AUCᵤ ratios in infected animals were not significantly different than those obtained in healthy rats (p>0.05, Wilcoxon test, R software) (Table 2).

TABLE 1

| | | C_{max,u} | t_{1/2,u} | CLᵤ | V_{SSu} |
|---|---|---|---|---|---|
| | | (ug/mL) | (min) | (mL/min/kg) | (mL/kg) |
| Healthy | Blood | 10.3 ± 2.6 | 78 ± 19 | 44.4 ± 5.1 | 3967 ± 557 |
| rats | Muscle | 8.6 ± 2.5 | 78 ± 9 | | |
| (n = 9) | Prostate | 8.2 ± 1.9 | 68 ± 11 | | |
| Infected | Blood | 12.3 ± 1.9 | 86 ± 23 | 35.8 ± 5.5 | 3344 ± 590 |
| rats | Muscle | 9.9 ± 3.8 | 77 ± 22 | | |
| (n = 11) | Prostate | 6.6 ± 2.7 | 83 ± 9 | | |

TABLE 2

| | AUC_{u muscle/blood} | AUC_{u prostate/blood} |
|---|---|---|
| Healthy rats (n = 9) | 1.06 ± 0.17 | 0.89 ± 0.17 |
| Infected rats (n = 11) | 1.03 ± 0.13 | 0.78 ± 0.21 |

In parallel to microdialysis, total PK parameters were obtained from total plasma concentrations and are presented in Table 3. The total plasma results are similar to microdialysis.

TABLE 3

| | CL_{t,plasma} | V_{d} | t_{1/2} | AUC |
|---|---|---|---|---|
| | (mL/min/kg) | (mL/kg) | (min) | (ug · min/h) |
| Healthy rats (n = 9) | 50.3 ± 7.1 | 4256 ± 851 | 72.0 ± 11.4 | 405 ± 57 |
| Infected rats (n = 11) | 45.6 ± 12.8 | 4016 ± 997 | 76.5 ± 9.9 | 472 ± 143 |

### Example 2

The present study (NCT04484740) is to determine concentrations of gepotidacin in plasma, prostate and tonsillar tissue of patients undergoing radical prostatectomy (RPE) for localized prostate, simple prostatectomy (PE) for benign prostate hyperplasia (BPH) or tonsillectomy (TE).

A single oral dose of 1500 mg gepotidacin will be administered to patients who will undergo radical prostatectomy (RPE) or simple prostatectomy (PE) and patients undergoing tonsillectomy (TE). The individual time-points of gepotidacin administration will be chosen to ensure that the time-point of tissue removal corresponds with one of six different sampling time-points, as closely as possible. After study drug administration RPE or TE will be performed according to clinical routine.

Subsequently, microdialysis (MD) probes will be inserted in the removed tissue (tonsillar or prostate tissue) ex-vivo and MD will be performed to determine unbound drug concentrations in the tissue.

Plasma PK samples will be collected just before study drug administration and up to 48 h after administration of gepotidacin.

Since MD provides the concentration of the unbound fraction of gepotidacin, for comparison we will calculate the unbound fraction of the concentration values obtained through blood sampling. To this end, we will determine the protein binding using ultrafiltration for each subject at the time-point closest to the Cmax. The individual protein binding can then be used to calculate the unbound plasma fraction of gepotidacin. This will allow to transform the plasma PK data to the same scale as the microdialysis data.

Samples will be analysed using non-compartmental analysis (NCA) for plasma concentrations and population pharmacokinetic models (PopPK) for tissue concentrations pooled with plasma concentrations.

### Example 3

The present study (NCT04484740) determined concentrations of gepotidacin in plasma, prostate and tonsillar tissue of patients undergoing radical prostatectomy (RPE) for localized prostate, simple prostatectomy (PE) for benign prostate hyperplasia (BPH) or tonsillectomy (TE).

A single oral dose of 1500 mg gepotidacin was administered to patients who were scheduled to undergo radical prostatectomy (RPE) or simple prostatectomy (PE) and patients undergoing tonsillectomy (TE). The individual time-points of gepotidacin administration were chosen to ensure that the time-point of tissue removal corresponds with one of six different sampling time-points up to 24 h, as closely as possible. After study drug administration RPE or TE was performed according to clinical routine.

Subsequently, microdialysis (MD) probes were inserted in the removed tissue (tonsillar or prostate tissue) ex-vivo and MD was performed to obtain samples for the determination of unbound drug concentrations in the tissue.

Blood samples for plasma PK were collected just before study drug administration and up to 48 h after administration of gepotidacin. Since MD samples provided the unbound gepotidacin concentrations, for comparison plasma PK was adjusted based on the measured in vitro unbound fraction.

Plasma PK parameters were obtained using non-compartmental analysis (NCA), whereas tissue PK parameters were obtained using a population pharmacokinetic modeling approach (PopPK) that incorporated plasma and tissue concentrations.

### Data Analysis

### Available Data

47 patients had assayable plasma concentrations, 29 were from the prostate cohort and 18 from the tonsil cohort. 24 patients from the prostate cohort had at least 1 assayable tissue sample, 11 patients from the tonsil cohort had at least 1 assayable tissue sample. In total 522 plasma samples, 94 prostate samples and 25 tonsil samples were collected.

1. A method for treating bacterial prostatitis in a human in need thereof, comprising administering to said human a therapeutically effective amount of gepotidacin or a pharmaceutically acceptable salt thereof.

2. The method as claimed in claim 1, wherein the bacterial prostatitis is acute prostatitis.

3. The method as claimed in claim 1, wherein the bacterial prostatitis is chronic prostatitis.

4. The method as claimed in claim 1, wherein the bacterial prostatitis is caused by bacteria selected from the group consisting of Escherichia coli, Pseudomonas aeruginosa, Klebsiella pneumoniae, Klebsiella oxytoca, Klebsiella aerogenes, Klebsiella variicola, Enterococcus faecalis, Enterococcus faecium, Enterococcus cloacae complex, Proteus mirabilis, Proteus hauseri, Proteus peneri, Serratia marcescens, Staphylococcus saprophyticus, Staphylococcus lugdenensis, Staphylococcus capitis, Staphylococcus caprae, Staphylococcus cohnii, Staphylococcus epidermidis, Staphylococcus haemolyticus, Staphylococcus hominis, Staphylococcus intermedius, Staphylococcus simulans Staphylococcus warneri, Streptococcus agalactiae, Streptococcus group F, Streptococcus group G, Streptococcus anginosus, Streptococcus australis, Streptococcus constellatus, Streptococcus cristatus, Streptococcus gordonii, Streptococcus infantarius, Streptococcus infantis, Streptococcus intermedius, Streptococcus massiliensis, Streptococcus mitis, Streptococcus oralis, Streptococcus mutans, Streptococcus parasanguinis, Streptococcus salivarius, Streptococcus sanguinis, Streptococcus vestibularis, Mycoplasma genitalium, Neisseria gonorrhoea, Salmonella spp, and Ureaplasma urealyticum.

5. The method as claimed in claim 1, wherein the bacteria is Escherichia coli.

6. The method as claimed in claim 1, wherein the gepotidacin is gepotidacin mesylate.

7. The method as claimed in claim 6, wherein the gepotidacin is gepotidacin mesylate dihydrate.

8. The method as claimed in claim 1, wherein the gepotidacin or a pharmaceutically acceptable salt thereof is administered orally.

9. The method as claimed in claim 1, wherein the human has failed at least one prior line of treatment for the bacterial prostatitis.

10-12. (canceled)
